(19) United States
(12) Patent Application Publication
(10) Pub. No.: US 2022/0208391 A1
(43) Pub. Date: Jun. 30, 2022

(54) SMART MASK

(71) Applicant: KING FAHD UNIVERSITY OF PETROLEUM AND MINERALS, Dhahran (SA)

(72) Inventors: Samir MEKID, Dhahran (SA); Kamel CHENAOUA, Dhahran (SA)

(73) Assignee: KING FAHD UNIVERSITY OF PETROLEUM AND MINERALS, Dhahran (SA)

(21) Appl. No.: 17/565,883

(22) Filed: Dec. 30, 2021

Related U.S. Application Data
(60) Provisional application No. 63/132,643, filed on Dec. 31, 2020.

(51) Int. Cl. G16H 50/70; G16H 50/80; G16H 50/20; G16H 10/60; G06F 1/16; A62B 18/08; A62B 18/02; H04W 4/80

(52) U.S. Cl. CPC G16H 50/70; G16H 50/80; G16H 50/20; H04W 4/80; G06F 1/163; A62B 18/088; A62B 18/025; G16H 10/60

(57) ABSTRACT

A smart mask including sensors, an RFID tag, a microcontroller and a communications device communicates by a near field communications protocol with a WiFi access point, which sends sensor readings, a location, and an identification to a cloud based smart mask monitoring application, which registers the smart mask, and stores the identification. The sensor readings are analyzed by the cloud based smart mask monitoring application to determine when a person wearing the smart mask may be contaminated by COVID-19. A neighborhood analysis is conducted to identify other smart mask wearers who may have come in contact with the contaminated person, and the other smart mask wearers are notified. Instructions are sent to the microcontroller to activate LEDs which indicate whether the health status of the person is normal, is possibly infected or is contaminated. A smart phone including a native smart mask monitoring application may display the health status.

### CROSS-REFERENCE TO RELATED APPLICATION

The present application claims benefit of priority to U.S. Provisional Application No. 63/132,643 having a filing date of Dec. 31, 2020 and which is incorporated herein by reference in its entirety.

### BACKGROUND

### Technical Field

The present disclosure is directed to a smart mask for protection from airborne disease contamination, such as droplets containing Covid-19 virus particles. The smart mask communicates by near field communications with a smart mask application stored in a cloud and a native smart mask application on a smart device.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

The World Health Organization (WHO) has provided guidelines (See: World Health Organization. (2020). “Advice on the use of masks in the context of COVID-19: interim guidance”, Apr. 6, 2020. World Health Organization, Doc. No. WHO/2019-nCov/IPC_Masks/2020.3), regarding the use of masks in communities, during home care, and in health care settings in areas that have reported cases of COVID-19. These guidelines are directed towards individuals in communities, public health and infection prevention and control (IPC) professionals, health care managers, health care workers (HCWs), and community health workers. The WHO report suggested that the two main routes of transmission of the COVID-19 virus are respiratory droplets and contact. Respiratory droplets are generated when an infected person coughs or sneezes. A person who is in close contact (within one to two meters) with someone who has respiratory symptoms (coughing, sneezing) is at risk of being exposed to potentially infective respiratory droplets. Droplets may also land on surfaces where the virus could remain viable; thus, the immediate environment of an infected individual can serve as a source of transmission (contact transmission). Touching the face after COVID-19 exposure to these droplets or touching the eyes, the nose or the mouth may infect the person with the COVID-19 virus. Therefore, it is necessary that at least medical personnel wear a full mask covering their entire face and breathe filtered air through the mask.

However, medical personnel, such as doctors, nurses and orderlies, during this pandemic, have suffered consequences from wearing masks for long, continuous hours, resulting in the examples of the damages shown on the faces in FIG. 1A and FIG. 1B. In FIG. 1A, a paper mask was worn for long hours by the subject. FIG. 1B points out the forehead and cheek abrasions resulting from wearing the paper mask.

A U.S. patent publication describing a face mask for protecting against dangerous disease, such as U.S. 2009/0255535A1, incorporated herein by reference in its entirety, describes a filtering face mask including at least one diagnostic device for identifying whether the wearer of the mask is infected with a contagious disease. The face mask includes an RFID tag and/or a bar code which can be read, and sensors, including a thermometer, an ear thermometer and a camera to identify and select potentially contagious individuals. The RFID tag can be associated with a bio-sampling device, which tests for disease. The bio-sampling data of the RFID tag can be communicated to a data aggregation center as the RFID tag is read. The filtering face mask has a transparent front shield and a filter, but is not a smart mask which communicates a communications packet through near field communications, such as Bluetooth and WiFi communications, nor does it have a microcontroller communicably connected to a communications device and the sensors, nor does it communicate with a smart mask application stored on a smart phone or by a cloud server.

In the current environment of COVID-19 disease, there is a need for better protection from exposure to airborne droplets carrying viral particles.

Accordingly, it is one object of the present disclosure to provide methods and systems for a smart mask which identifies viral contamination of a human and communicates the viral contamination through low power near field communications to an access point or smart phone, for transmission to a cloud based smart mask monitor.

### SUMMARY

In an exemplary embodiment, a smart mask, comprising a transparent acrylic shield having a large opening configured to fit against a face and a small opening opposite the large opening, an outer seal covering a circumference of the large opening; a nose cone gasket covering the small opening; a cylindrical air filter unit having a diameter configured to fit within the nose cone gasket, the cylindrical filter unit including a filter pocket; a replaceable membrane located within the filter pocket, wherein the replaceable membrane is configured to filter airborne droplets; an inner shield connected to the nose cone gasket, wherein the inner shield is configured to surround the nose and mouth of the human wearing the smart mask, and convey filtered air to the nose and mouth; a plurality of LED lights located on an exterior of the transparent acrylic shield; a plurality of sensors located on the transparent acrylic shield, each sensor configured to generate a sensor reading; a communications unit configured for near field communications; a global positioning system, GPS, unit configured to provide a geographic location of the smart mask; a microcontroller located on the transparent acrylic shield, wherein: the microcontroller is connected to the communications unit, the GPS unit and the plurality of sensors; the microcontroller further includes an RFID tag configured to generate an identification signal; the microcontroller is configured to generate a message including the sensor readings, the identification signal and the geographic location of the smart mask; the microcontroller is configured to transmit the message to the communications unit; and the communications unit is configured to transmit the communication packet using near field communication.

In aspects of the present disclosure, the airborne droplets are water droplets contaminated with COVID-19 virus particles, and the replaceable membrane is configured to filter the water droplets contaminated with the COVID-19 virus particles.

In an aspect of the present disclosure, a smart phone having a native smart mask monitoring application receives the communication packet from the communications unit by a Bluetooth protocol. The smart phone may communicate the communication packet over a mobile phone communications channel to the cloud based smart mask monitoring application stored by a cloud server.

In aspects of the present disclosure, a WiFi access node receives the communication packet from the communications unit over near field communications using a WiFi protocol. The WiFi access node may then communicate the communication packet by using a machine-to-machine Internet of Things (MQTT) protocol, to a cloud based smart mask monitoring application stored by a cloud server.

In another exemplary embodiment, a method for protecting humans from COVID-19 is described, comprising covering a face of a first human with a protective smart mask, wherein the protective smart mask is configured to filter air entering a mouth and nose of the face; sending, by a proximity sensor, a proximity signal to a microcontroller located on the protective smart mask when a proximity sensor detects an object within one meter of the first human; measuring, with an ear canal thermometer, an ear canal temperature of the first human, generating an ear canal temperature signal and transmitting the ear canal temperature signal to the microcontroller; detecting, with an accelerometer, rapid movements of the smart mask and sending an accelerometer signal to the microcontroller; detecting a face temperature by a contactless infrared temperature sensor located on the smart mask, and sending a face temperature signal to the microcontroller; generating, by a global positioning system, GPS, unit operatively connected to the microcontroller, a geographic location of the smart mask; generating, by an RFID tag operatively connected to the microcontroller, an identification signal; and transmitting, by a communications unit operatively connected to the microcontroller, a message including the proximity sensor signal, the ear canal temperature signal, the accelerometer signal, the face temperature signal, the geographic location and the identification signal by a near field communication protocol to a WiFi access point; and transmitting, by the WiFi access point, the message to a cloud server hosting a cloud based smart mask monitoring application using an using an MQTT protocol.

In another exemplary embodiment, a cloud based smart mask monitoring application configured to detect viral exposure of a human wearing a smart mask is described, comprising receiving, by the cloud based smart mask monitoring application, a communications packet including proximity sensor signals, ear canal temperature signals, accelerometer signals, face temperature signals, a geographic location signal and a smart mask identification signal; analyzing the communications packet to detect viral exposure of a first human wearing the smart mask; when detecting viral exposure of the first human: identifying, from the proximity sensor signals, whether objects within a proximity of the protective face are one or more second humans; generating an analysis report of the viral exposure of the first human; transmitting the report to a native face mask application stored on a smart phone of the first human; and alerting the one or more second humans of the danger of viral contamination by one or more of calling the one or more second humans, texting the one or more second humans, emailing the one or more second humans, and activating a lighting indicator on the smart mask worn by the first human.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is photograph of damage due to wearing a face mask, according to certain embodiments.

FIG. 1B is photograph of forehead and cheek abrasions due to wearing the face mask of FIG. 1A, according to certain embodiments.

FIG. 2A is an illustration of the placement of a smart mask upon a face, according to certain embodiments.

FIG. 2B is an illustration of the smart mask, according to certain embodiments.

FIG. 2C is a block diagram of communications environment of the microcontroller of the smart mask, according to certain embodiments.

FIG. 2D is a block diagram of the microcontroller of the smart mask, according to certain embodiments.

FIG. 3 is an overview of a system using the smart mask, according to certain embodiments.

FIG. 4A is an example of an infrared temperature sensor, according to certain embodiments.

FIG. 4B is an example of a tympanic ear canal temperature sensor, according to certain embodiments.

FIG. 4C is an example of an accelerometer circuit board, according to certain embodiments.

FIG. 4D is an example of an accelerometer, according to certain embodiments.

FIG. 4E is an example of a proximity sensor, according to certain embodiments.

FIG. 5A is an example of a bar code, according to certain embodiments.

FIG. 5B is an example of an RFID tag, according to certain embodiments.

FIG. 6 is an exemplary flowchart for registering the smart mask with the cloud based smart mask monitoring application, according to certain embodiments.

FIG. 7 is an exemplary flowchart for cloud based smart mask monitoring of temperature data and indicator control, according to certain embodiments.

FIG. 8 is an exemplary flowchart for cloud based smart mask monitoring of proximity and indicator control, according to certain embodiments.

FIG. 9 is an exemplary flowchart for processes of the cloud based smart mask monitoring application, according to certain embodiments.

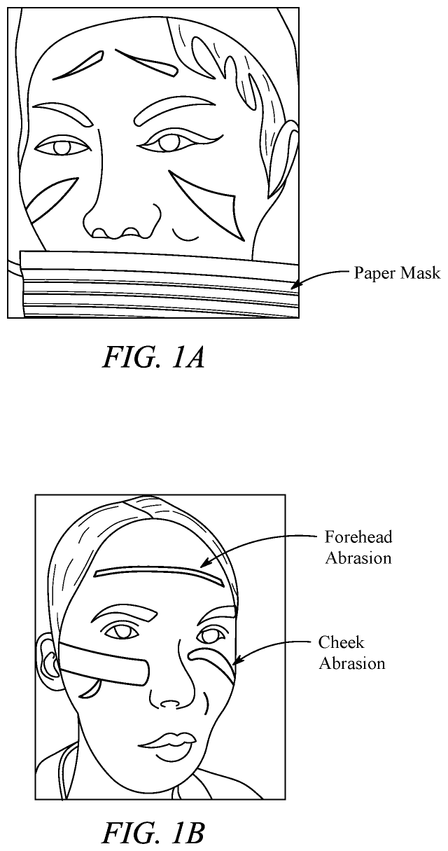

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a smart mask, a method for protecting humans from COVID-19, and a mobile cloud application configured to detect viral exposure of a human wearing a smart mask.

The smart mask includes a transparent acrylic shield having a filter pocket for holding a surgery-grade replaceable filter. A plurality of sensors located on the smart mask is configured to generate sensor readings. The smart mask includes a microcontroller operatively connected to the plurality of sensors. The microcontroller is operatively connected to a communications device configured for near field communications, wherein the near field communications include Bluetooth and WiFi communications. The microcontroller is configured to collect sensor readings from the plurality of sensors and the communications device is configured to transmit the sensor readings by near field communication to one of a wireless router (WiFi access point) and a smart phone. The wireless router transmits the sensor readings, along with an identification of the smart mask, to a cloud based smart mask monitoring application.

Embodiments to a method for protecting a human face from airborne disease particles and a cloud based smart mask monitoring application configured to detect viral exposure are described.

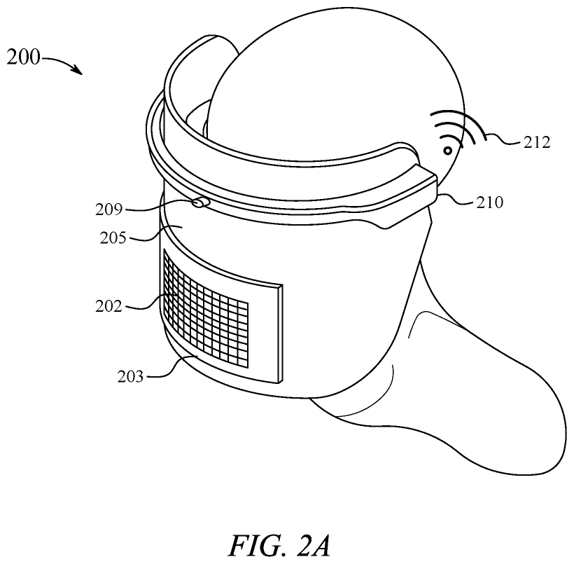

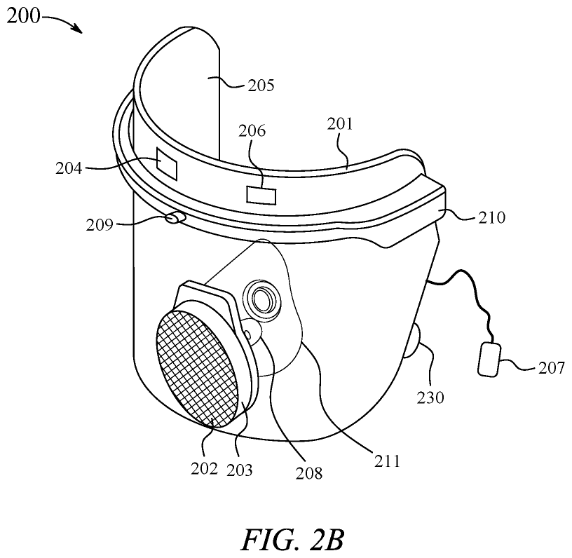

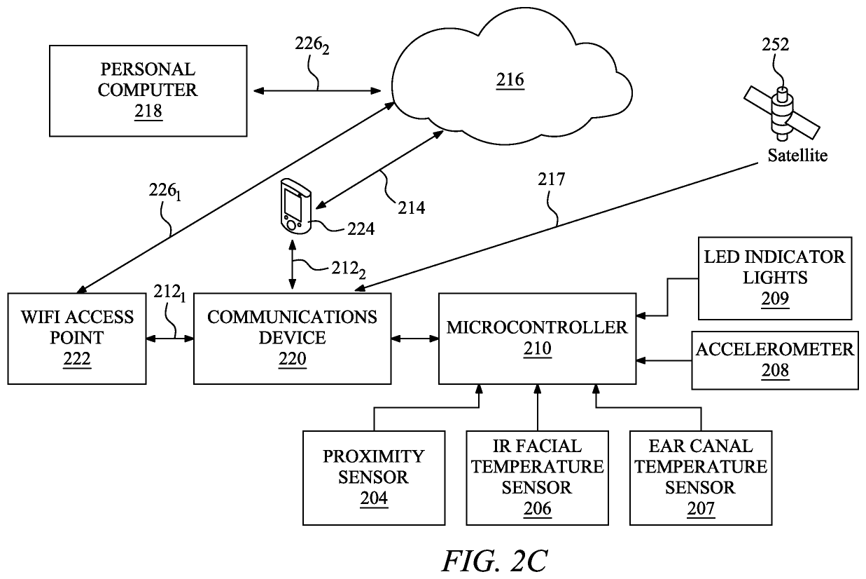

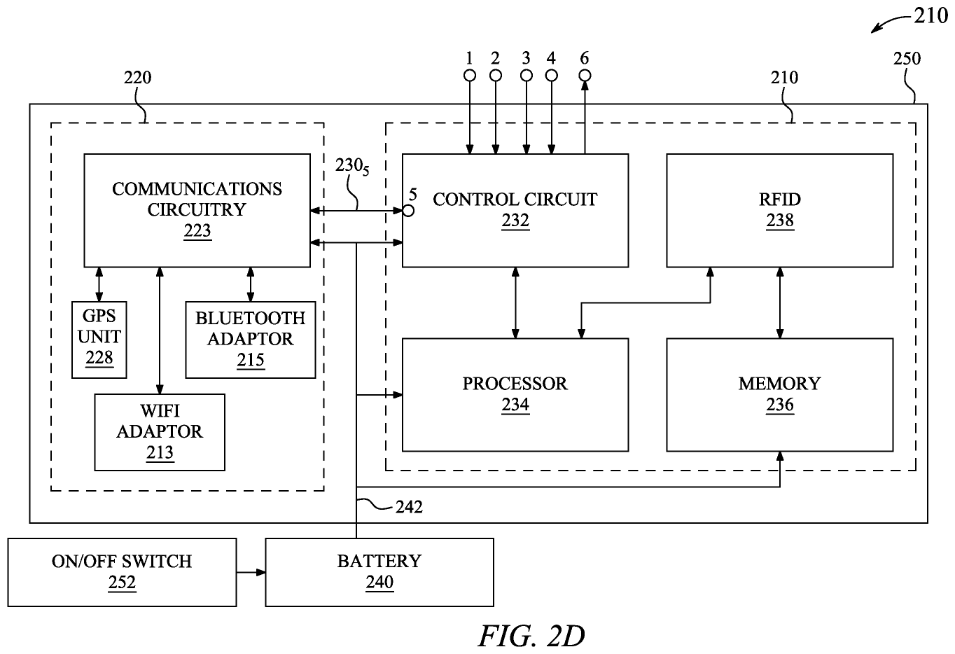

FIG. 2A and FIG. 2B illustrate aspects of a smart mask 200. As shown in FIG. 2A and FIG. 2B, the smart mask 200 includes a transparent acrylic shield 205 which covers the face of a human while sealing the nose and mouth with a surgery-grade replaceable filter 202 inserted in a filter unit 203 of the smart mask 200. The filter unit 203 holds the filter and an inner silicone mask 211 which seals against the nose and face of a human wearing the smart mask. FIG. 2A shows a prototype of the smart mask installed on the face of a mannequin representing a human head. The smart mask 200 may be held to the face by a strap (not shown) which goes around the head of the human above the ear and connects to either side of the edges of the smart mask.

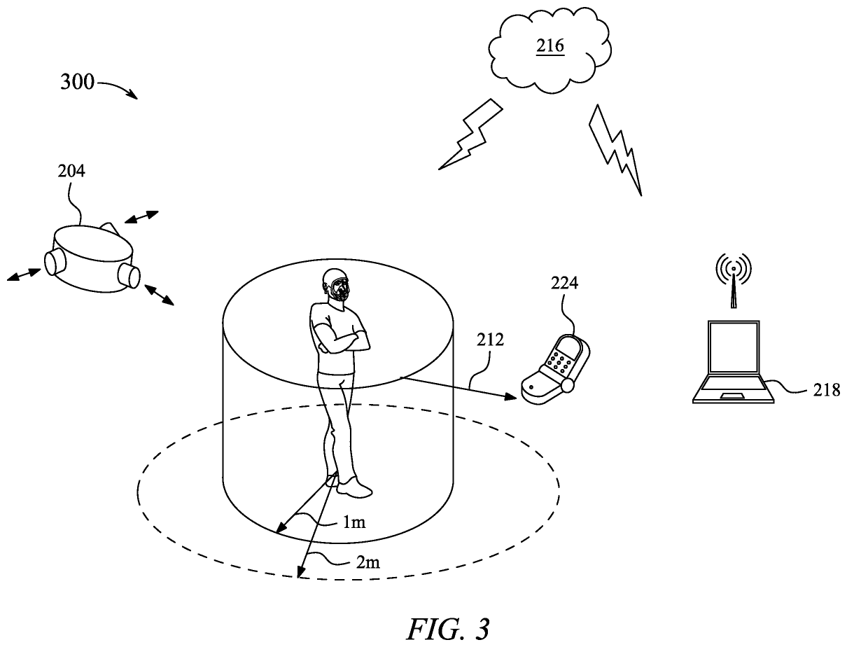

As shown in FIG. 2A, the smart mask 200 may include a microcontroller 210 and wiring band which includes LED indicator lights 209. In FIG. 2A, at least the upper half of the smart mask is transparent. The microcontroller 210 of the smart mask is connected to or equipped with a communications device 220 (see FIG. 2B) which communicates with a wireless router, such as a WiFi access point by near field communications 212. The wireless router may also communicate with a smart phone 224 (see FIG. 3) of the human wearing the smart mask 200, by Bluetooth communications.

As shown in FIG. 2B, the smart mask may include a proximity sensor 204, an infrared temperature sensor 206 connected to LED indicator lights and a microcontroller 210. The infrared temperature sensor 206 transmits temperature readings to the microcontroller. The infrared temperature sensor 206 may activate the LED indicator lights based on the temperature reading. The microcontroller performs further processing which includes sending the temperature readings and other sensor readings to a cloud based smart mask monitoring application 216 (see FIG. 2C), from which it receives instructions to light LED indicator lights according to a pattern based on additional processing. The smart mask may also contain a QR code 230 at any location on the filter unit 203, the seal 201 or the transparent acrylic shield 205, as long as it is placed out of the line of sight of the human. An example of a QR code is shown in FIG. 5A. The QR code is a type of two-dimensional barcode, which is a machine-readable optical label that can contain information about the item to which it is attached. The QR code may contain data for at least one of a locator, an identifier, or a tracker that points to the cloud based smart mask monitoring application 216. The QR code consists of black squares arranged in a square grid on a white background, which can be read by an imaging device such as a camera of a smart phone, and processed, by a microprocessor of the smart phone, using Reed-Solomon error correction until the image can be appropriately interpreted. The required data is then extracted from patterns that are present in both horizontal and vertical components of the image. The smart phone may include a native smart mask monitoring application which is linked to the cloud based smart mask monitoring application 216. The smart mask may transmit the QR code to the cloud based smart mask monitoring application 216 to register the smart mask 200.

FIG. 2B illustrates the smart mask 200 and further features of the smart mask.

In a non-limiting example, the transparent acrylic shield 205 may be made of plexiglass. In a further non-limiting example, the transparent acrylic shield 205 may be made of Perspex®, manufactured by PERSPEX International, Ltdl, 75 Duckworth St, Darwen BB3 1QB, United Kingdom. Preferably, the acrylic shield is hydrophobic, so that airborne water droplets do not adhere to the interior of the smart mask. Hydrophobicity may be imparted to the acrylic shield by coating the interior with a layer of graphene.

The transparent acrylic shield 205 may form a cone shape, the cone shape having a large opening configured to fit against the face and a small opening opposite the large opening, the small opening forming the filter unit 203 being oval as shown in FIG. 2B, or may have the straight shield shown in FIG. 2A. The form of the smart mask is not limited to a straight shield or a cone shaped field, and may have any shape which serves to protect the wearer from airborne disease droplets. The transparent acrylic shield 205 may include a seal 201 covering a circumference of the large opening, and the filter unit 203 covering a circumference of the small opening, wherein the filter unit 203 includes a filter pocket for inserting the surgery-grade replaceable filter 202 which has a diameter configured to fit within the filter pocket. The cylindrical filter unit includes a replaceable filter membrane.

The seal 201 edges the transparent acrylic shield 205 where it contacts the face. The seal 201 is designed to seal the face of the human from air which does not pass through the filter. The seal 201 is soft and biocompatible, so it does not damage the face. The seal 201 may be selected from any of a silicone seal and a polymer channel filled with silicone gel. The seal 201 may be able to be sterilized or removed and replaced in order to protect the wearer from an accumulation of disease particles.

For illustrative purposes, the proximity sensor is shown mounted at the top of the smart mask, but may be located anywhere on the seal 201 or the nose cone 203.

The contactless infrared temperature sensor 206 connected to the LED indicator lights 209 is mounted on the transparent acrylic shield 205. The infrared circuitry may be located on the interior of the transparent acrylic shield 205 above the forehead of the human, and the LED lights may extend through the transparent acrylic shield 205 to the exterior of the transparent acrylic shield 205, so that the lights may be viewed without distortion by the curvature of the shield. The LED lights are connected to the microcontroller 210 and may additionally be turned on or off based on proximity sensor and accelerometer readings.

In a non-limiting example, the surgery-grade replaceable filter 202 may be an N95 filter. An N-95 filter refers to a type of disposable particulate respirator. Particulate respirators protect by filtering particles out of the air as a person breathes. These respirators protect against airborne biological agents such as bacteria and viruses. By definition, an N95 filter filters at least 95% of airborne particles. In another non-limiting example, the surgery-grade replaceable filter 202 may be an N99 filter, which filters at least 99% of airborne particles. In a further non-limiting example, the surgery-grade replaceable filter 202 may be an N100 filter, which filters greater than 99.97% of airborne particles. (See: Robinson, P., “Comparison of Mask Standards, Ratings, and Filtration Effectiveness”, Smart Air, Jul. 27, 2021, 901-B, Huapu Huayuan, 9 Dongzhimen Nan Da Jie, Dongcheng District, 100007, Beijing, China, incorporated herein by reference in its entirety).

In a non-limiting example, the acrylic shield may be made of plexiglass. In a further non-limiting example, the acrylic shield 205 may be made of Perspex®, manufactured by PERSPEX International, Ltdl, 75 Duckworth St, Darwen BB3 1QB, United Kingdom. Preferably, the acrylic shield is hydrophobic, so that airborne water droplets do not adhere to the interior of the smart mask. Hydrophobicity can be imparted to the acrylic shield by coating the interior with a layer of graphene.

For illustrative purposes, the proximity sensor is shown mounted at the top of the smart mask, but may be located anywhere on the edge of the transparent acrylic shield 205 or the filter unit 203.

As shown in FIG. 2A and FIG. 2B, the contactless infrared temperature sensor 206 is connected to the LED indicator lights 209 which indicate progressive changes in the temperature of the face through a range. There may be at least three LEDs in the colors of red, yellow and green.

The smart mask 200 includes a tympanic ear canal temperature sensor 207.

The smart mask further includes a proximity sensor 204 which detects other humans within a set distance. The proximity sensor facilitates social distancing by buzzing or otherwise sounding an alarm to notify the human wearing the face mask of other humans within the set distance. In a non-limiting example, the set distance may be less than or equal to one meter. In another non-limiting example, the set distance may be less than or equal to two meters.

The smart mask further includes an accelerometer 208. The accelerometer is configured to sense movements which indicate coughing or sneezing of the human wearing the smart mask. The accelerometer may be mounted on one of the interior and the exterior of the smart mask on the filter unit 203.

The smart mask further includes the microcontroller 210. The microcontroller 210 is operatively connected to the proximity sensor 204, the tympanic ear canal temperature sensor 207, the accelerometer 208, and the infrared temperature sensor 206 to receive sensor readings from the sensors 204, 207, 208 and 206. The microcontroller may average the readings of the tympanic ear canal temperature sensor 207 and the infrared temperature sensor 206, compare the average to temperature setpoints, and send control signals to the LED indicator lights 209 to light up in a pattern indicated by the comparison results. 37.5° C.-38.5° C. (99.5° F. to 101.3° F.). Actuation of a green LED indicates that the average temperature is normal (below 37.5° C.). Actuation of a yellow LED indicates that the average temperature is greater than or equal to 37.5° C. and less than 38° C. Actuation of a red LED indicates that the average temperature is greater than or equal to 38° C. (100.4° F.). An average temperature greater than or equal to 38° C. indicates that immediate action should be taken to remove the human wearing the smart mask 200 from proximity with others. The microcontroller 210 may control the LED indicator lights based on internal processing, and update the status of control signals to the LED indicator lights after receiving instructions from the cloud based smart mask monitoring application 216, wherein the instructions are based on further processing.

FIG. 2C and FIG. 2D illustrate the microcontroller 210 connected into a circuit board 250 which includes a communications device 220. The microcontroller is connected by circuit board wiring to the proximity sensor 204, the tympanic ear canal temperature sensor 207, the accelerometer 208, and the infrared temperature sensor 206. The microcontroller is further connected by circuit board wiring to the communications device 220, which may be installed on the circuit board. As shown in FIG. 2D, the microcontroller 210 circuit includes an RFID tag 238, control circuit 232, a processor 234 and a memory 236. The control circuit 232 has pins 1, 2, 3, 4 and 5. Pin 1 connects to the proximity sensor 204 to receive proximity sensor readings, pin 2 connects to the infrared temperature sensor 206 to receive forehead temperature readings, pin 3 connects to the tympanic ear canal temperature sensor 207 to receive ear canal temperature readings, pin 4 connects to the accelerometer to receive accelerometer readings. Pin 6 connects to the LED indicator lights 209.

Although not shown in FIG. 2D, the proximity sensor 204, the tympanic ear canal temperature sensor 207, the accelerometer 208, and the infrared temperature sensor 206 may each contain internal circuitry which generates the respective readings.

Microcontroller pin 5 connects the microcontroller to the communications device 220.

The communications device is configured for near field communication 212₁ with any wireless router or WiFi access point 222 in range. Near field communication with a wireless router uses frequencies of 2.4, 3.6 and 5 GHz, for a distance less than or equal to 92 meters. Near field communications with a wireless router are generally referred to as WiFi communications. WiFi is a family of wireless network protocols, based on the IEEE 802.11 family of standards, which are commonly used for local area networking of devices and Internet access, allowing nearby digital devices to exchange data by radio waves. The WiFi connection of the present disclosure uses the IEEE 802.11b and/or 802.11a/g standard as a link to the WiFi access point. The WiFi access point transmits the sensor readings, the location and the identification of the smart mask over a MQTT protocol to the cloud based smart mask monitoring application, since the messages to be transmitted are of minimal size. MQTT is a machine-to-machine (M2M) Internet of Things connectivity protocol, a lightweight transfer protocol used for communication between small IoT enabled devices, such as the smart mask 200. Alternatively, the message may be formulated as a communications packet, which is sent by the WiFi router to the cloud based smart mask monitoring application 216 over an RF communication channel.

As shown in FIG. 2C and FIG. 2D, the communications device 220 receives the sensor readings from the microprocessor 210, an identification signal generated by RFID tag 238, and a GPS location of the smart mask generated by the GPS unit 228 (FIG. 2D). The GPS unit 228 of the communications device 220 receives location information of the smart mask 200 from the satellite 252 over communications path 217. An example of an RFID tag is shown in FIG. 5B.

The communications device transmits the sensor readings, location and identification of the smart mask to the WiFi access point 222 over WiFi communications channel 212₁. The WiFi access point 222 then transmits the sensor readings, location and identification of the smart mask using the MQTT protocol over communications channel 226₁.

The communications device 220 is further configured to communicate the sensor readings, location and identification of the smart mask 200 using near field communications to a smart phone 224 of the human wearing the smart mask. The smart phone may include a native smart mask monitoring application. The native smart mask application may perform some or all of the monitoring tasks performed by the cloud based smart mask monitoring application stored in cloud 216. The native smart mask monitoring application may handle the tasks of registering the smart mask 200 with the cloud based smart mask monitoring application 216. Near field communications with the smart phone 224 use a Bluetooth protocol at 2.4 GHz at low power over a short range (usually less than 10 meters). The power needed for WiFi communications is greater than power needed for Bluetooth communications. The smart phone may communicate a message containing the sensor readings, location and identification of the smart mask to the cloud based smart mask application 216 over an LTE communications path 214. The smart phone may use 4G LTE or 5G LTE to connect to the cloud based smart mask application 216. The native smart mask monitoring application may perform some or all of the processing needed to analyze the sensor readings and send the results as the instructions directly to the communications device 220. The native smart mask monitoring application may send the results of its processing to the cloud based smart mask application 216 to include in the cloud memory of the cloud based smart mask application 216, and may receive updates from the cloud based smart mask application 216.

The cloud based smart mask monitoring application is configured to use processors, controllers, memories, databases stored by a cloud server. A cloud server is a pooled, centralized server resource that is hosted and delivered over a communications network and accessed on demand by multiple users. Cloud servers can perform all the same functions of a traditional physical server, delivering processing power, storage and applications.

The cloud based monitoring application 216 may receive the message from the WiFi access point, match the identification to an identification stored in a smart mask database, which includes a registration of the smart mask and a user profile of the human who owns the smart mask. The cloud based monitoring application 216 may store the message by its location in a memory associated with the cloud based monitoring application 216. The cloud based monitoring application 216 determines from the sensor readings a probability that the human wearing the smart mask is infected with COVID-19. When the probability is high (greater than 60%) that the human is infected with COVID-19, the cloud based monitoring application 216 searches the memory for other smart masks which have been within a proximity distance of one meter of the human wearing the smart mask. The cloud based monitoring application 216 may then contact the owners of the other smart masks to inform them that they have been exposed to COVID-19. Additionally, the cloud based monitoring application 216 transmits a signal through the WiFi access point or to the smart phone 224, then through the near field communication paths to the microprocessor to light the appropriate LED light indicating the infection status of the human wearing the smart mask.

The cloud based monitoring application 216 stores the messages (ID, location and temperature) from each smart mask user in the cloud memory.

The data is updated regularly on the cloud for variety of purposes. The human wearing the smart mask can be informed about his temperature history, exposure to others wearing a smart mask, and his location. Large data from all smart mask wearers is used for further analysis in defining possible coronavirus contamination and the history of the smart mask wearers.

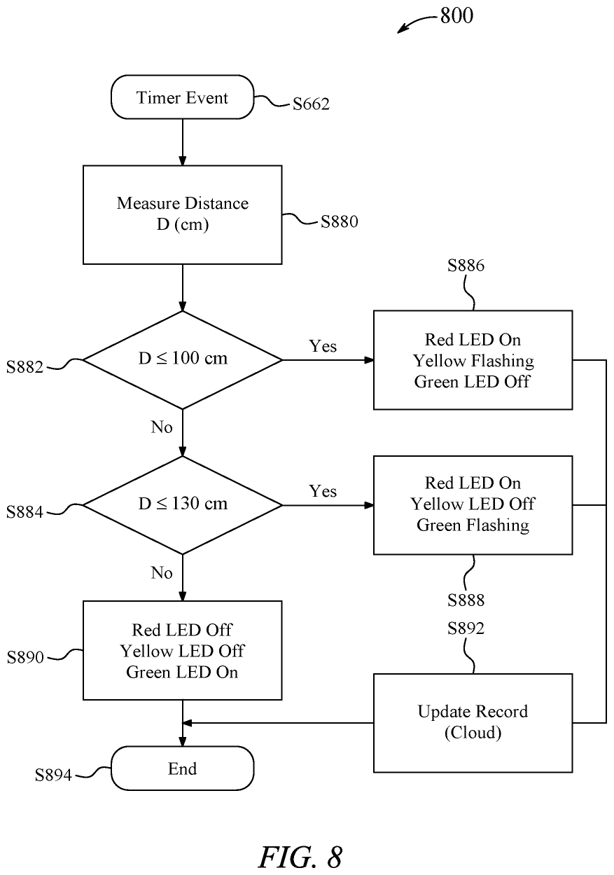

FIG. 3 illustrates a human wearing the smart mask 200 and the procedures regarding the proximity sensor 204. When the proximity sensor reading indicates another person within 2 meters of the human wearing the smart mask, a Bluetooth wireless connection may be made to the smartphone of the human, which includes a native smart mask monitoring application installed upon it. The smart phone may analyze the proximity data in a similar manner as the cloud based monitoring application 216 does in FIG. 8. The smart phone may transmit a Bluetooth message to the microprocessor 210 to light the appropriate LED based on the analysis. The smart phone may then transmit updated proximity readings to the cloud based monitoring application 216 over LTE as described above. The cloud based monitoring application 216 may in turn send a report of the smart mask readings to a personal computer 218 of the human wearing the smart mask.

Manufacturing of the smart mask 200 incorporates international health requirements for the filter type. Initial prototypes can be made with 3D printing but mass production will need manufacturing through injection molding for example with a die design. Cost is expected to be $10-$15 per mask in mass production.

The filter may include features such as changing color when dirty or wet, to indicate that it is time to change the filter. The filter should be visually inspected before wearing the smart mask to ensure that the filter does not need to be replaced.

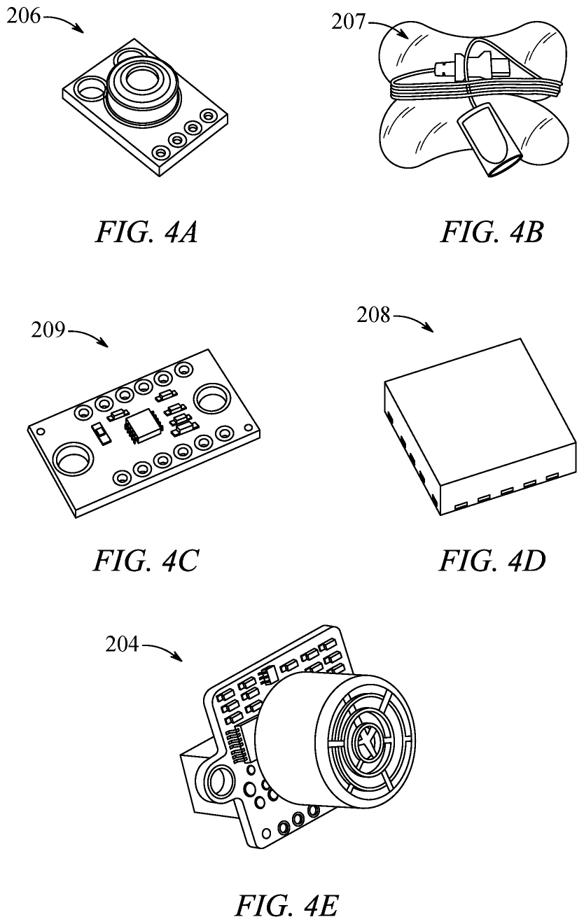

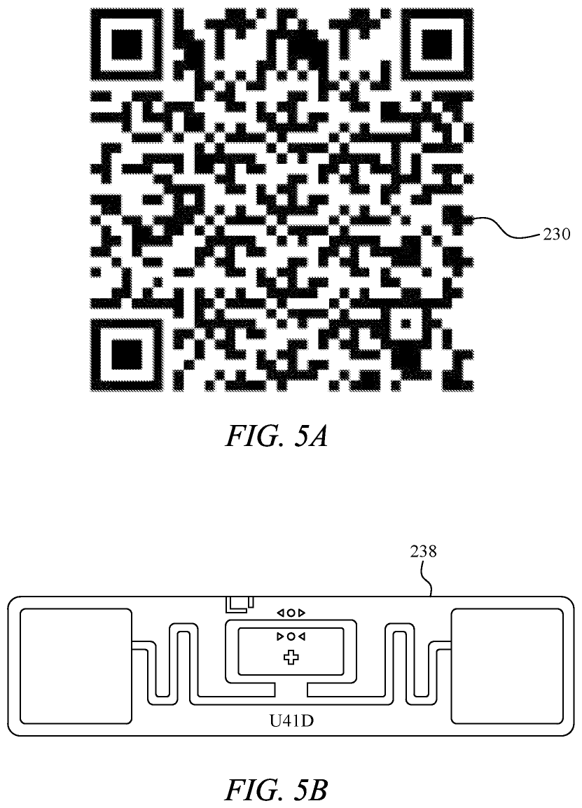

FIG. 4A shows a non-limiting example of a contactless infrared temperature sensor 206 which may be used in the smart mask 200. The contactless infrared temperature sensor has a high resolution of plus/minus 0.1° C. It is connected to the microprocessor by an I2C connection, which is a low power, serial connection for two integrated circuits.

FIG. 4B shows a non-limiting example of a tympanic ear canal temperature sensor 207 which may be used in the smart mask 200. The tympanic ear canal temperature sensor 207 has a high resolution of plus/minus 0.1° C. It is connected to the microprocessor by an I2C connection.

FIG. 4C shows a non-limiting example of an LIS3DH, Triple Axis Accelerometer Breakout Board 209 which connected the accelerometer 208 to the microprocessor which may be used in the smart mask 200. LIS3DH accelerometer is a low power sensor well suited to detect rapid changes in movement.

FIG. 4D shows a non-limiting example of a 3-Axis MEMS Accelerometer—MMA8452Q which may be used with the breakout board 209 of FIG. 4C.

FIG. 4E shows a non-limiting example of the proximity sensor 204 used in the smart mask prototype.

In aspects of the present disclosure, the smart mask is design to protect the user against contamination and to monitor at least one vital sign commonly indicating possible contamination.

The smart mask 200 has the following characteristics:

1—Unique tag number and registration using a user ID

2—a protective comfortable mask covering full face against COVID-19 Pandemic

3—an interchangeable air filter

4—the air filter is sealed to nose and mouth.

5—data is sent wirelessly to a cloud based smart mask monitoring application.

6—The data includes:
a. Human body temperature.
b. Geographic location.
c. Proximity distance to meet social distancing requirements.
d. Accelerometer readings indicating possible coughing/sneezing.

7—The data are processed in the cloud based smart mask monitoring application 216.

8—Data processing is performed by the cloud based smart mask monitoring application 216 to find potential COVID-19 contamination or other flu viruses.

9—When COVID-19 contamination is found, proximity data is analyzed to identify the historical neighborhood and other potentially contaminated people.

10—Actions include calling contaminated people to alert them that they need to be tested.

11—A native smart mask monitoring application is used to register the smart mask and link the smart mask data to the cloud based smart mask monitoring application 216.

The electric block circuit of the smart mask was designed around an AVR Atmega microcontroller. This microcontroller was equipped with a WiFi module, and has the following features: low power consumption, necessary serial interface protocols: I2C, SPI and RS232, analog to digital converters for reading analog data and a wireless communication module.

Each smart mask, is referred to as a node in the system, or IoT device, and has its own unique ID and is independent of other nodes in the network. Nodes are tagged using RFID or QR codes. All the smart masks communicate with the server of the cloud based smart mask monitoring application 216 using available routers and access points, which may include a user's smart phone. For security purposes, each node device (smart mask 200) is assigned an access token (including the QR code and/or the RFID identification code) to grant it access to the cloud based smart mask monitoring application 216. Short packets of data sent to the cloud based smart mask monitoring application 216 include all sensor readings.

During manufacture, each smart mask is assigned a tag number using a tagging system. When masks are dispensed to the smart mask wearers, a tag reader reads the identification of the smart mask from the QR code and the smart mask identification is registered with the cloud based smart mask monitoring application. The tag reader may be a scanner of a smart phone of the smart mask user.

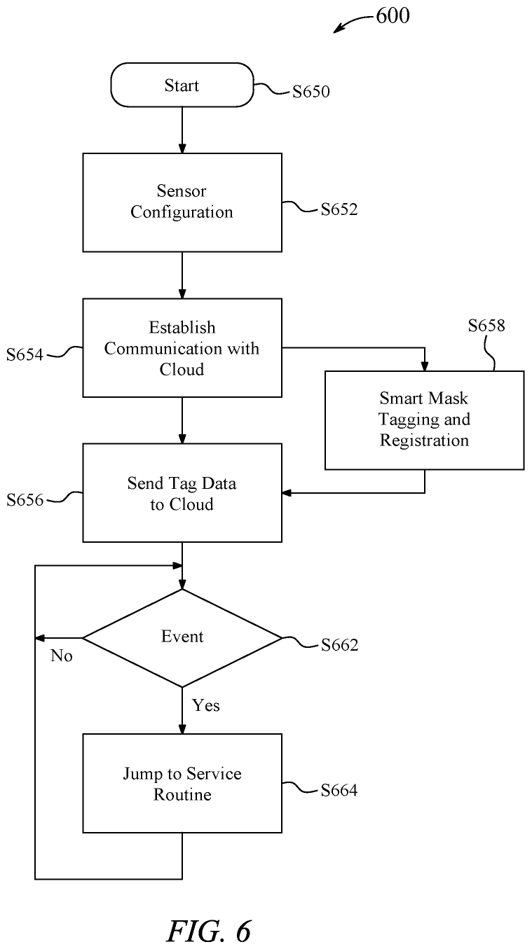

As shown in the flowchart of FIG. 6, the smart mask system is event driven and modular. The modularity of the physical system is reflected in the software design. At initialization S650, the system checks the different modules, such as the sensors and communications device and at S652 performs start up configurations for proper functioning. At S654, a connection with the cloud based smart mask monitoring application is established, and at S658 security information is exchanged to link the QR tag, RFID identification and user information to the cloud based smart mask monitoring application 216. After the smart mask is set-up, the microprocessor 210 and communications device 220 go into deep sleep mode, thus reducing power consumption to few μA. The microprocessor 210 is periodically woken up by its internal real time clock to receive readings from the sensors. Sensor readings are taken every 20 minutes to 30 minutes, to update data and extend battery life. Each time the system is active, readings are taken, the WiFi module is enabled and at S656 data is sent to the server of the cloud based smart mask monitoring application 216.

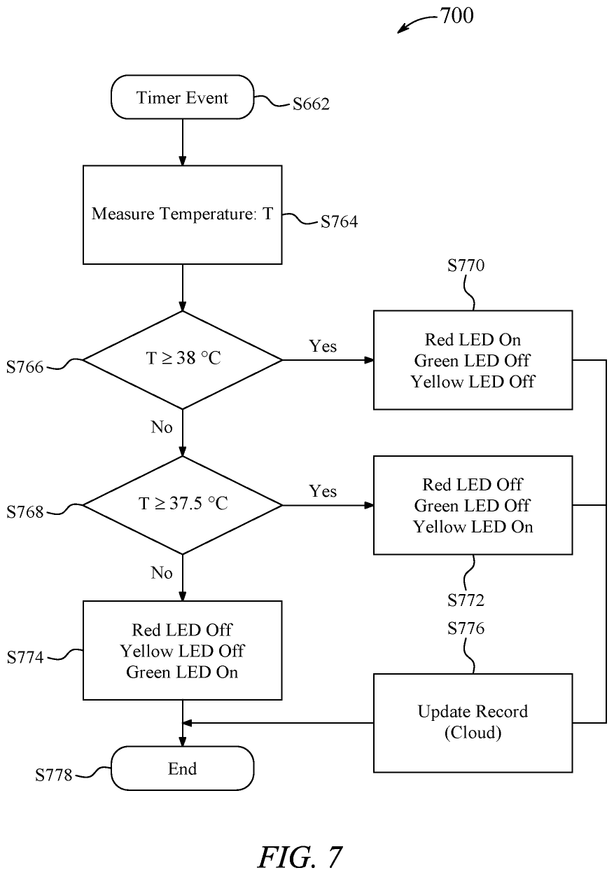

FIG. 7 shows a flow chart showing the routine used to determine which LED light should be lit. At S662, a timer event registers when the wake up timer of the microcontroller 210 wakes up the system. Temperature readings are checked against a series of temperature threshold values in the range of 37.5° C.-38.5° C. (99.5° F. to 101.3° F.). At S764, when an event occurs in which the temperature threshold of 38° C. is reached, a service routine S664 is started in which a red LED indicator is lit at S770, with the green and yellow LEDs off, alarming the person wearing the mask as well as showing others near him that he may be contaminated with COVID-19. When the temperature reading has not reached 38° C., but is greater than or equal to 37.5° C., the yellow LED is turned on, with the red and green LEDs off at S772, indicating the person wearing the mask may be getting sick. If the temperature reading is less than 37.5° C., the green LED is turned on, with the red and yellow LEDs off, indicating the person has a normal temperature. At S776, the comparisons at S770 and S772 are updated to the cloud based smart mask monitoring application.

FIG. 8 is a flowchart of the analysis of the proximity sensor 204 readings. Distance measurements are taken periodically and appropriate alarms and update of data at the cloud level are performed. When a timer event S662 wakes up the microprocessor to take readings, at S880 the proximity sensor measures the distance in centimeters to any other person within the range of one to two meters. The microprocessor is configured to light the LEDs in patterns which indicate the distance to other persons and warn the wearer of the smart mask and other persons near him that social distancing is not achieved. At S882, if it is determined that the distance is less than or equal to 100 cm, at S886 the red LED is turned on and the yellow LED flashes. The green LED is off. At S884, when the distance is determined to be greater than 100 cm but less than or equal to 130 cm, at S888 the red LED is turned on, the yellow LED is off, and the green LED flashes. At S892, the comparison data of S886 and S888 is updated at the cloud based smart mask monitoring application 216. At S890, when the distance is determined to be within the social distancing requirements, the red and yellow LEDs are turned off and the green LED is turned on. At S894, the routine ends until the next timer event. In a non-limiting example, a proximity distance threshold may be set to one meter. In another non-limiting example, the proximity distance threshold may be set to two meters.

When the accelerometer 208 detects an unusual movement, it fires up an interrupt and awakens the microcontroller unit 210, which assesses the strength of the movement and records it. If the movement is repetitive, such as sneezing and/or coughing, the microprocessor triggers an alarm. The alarm is a flashing red LED. A message is sent to the cloud based smart mask monitoring application 216 or to the smart phone 224 of the human wearing the smart mask for analysis of the repeated accelerometer readings. Events S662 which activate the microprocessor could be an internal timer event (FIG. 7), which wakes up the IoT node periodically, or a signal coming from an external device, such as the accelerometer indicating the detection of a sudden movement.

The accelerometer is put on wake mode continuously and is set to send a signal to the microcontroller upon registering a sudden movement. The accelerometer readings are analyzed in a similar manner as the temperature readings (FIG. 7) and proximity sensor readings (FIG. 8), and an appropriate LED is turned on as described above. The accelerometer must be calibrated to determine the magnitudes of the sensor readings which indicate coughing and sneezing.

The cloud based smart mask monitoring application 216 is hosted in a cloud based application server, which may be dedicated to host services and functions that may be executed by the cloud based smart mask monitoring application. Additionally, the application server may store information regarding the smart mask wearer, historical temperature readings, historical locations, and other details, related to the use of the smart mask 200. With the capability of such storage in the application server, the cloud based smart mask monitoring application may fetch additional data from the application server relating to the smart mask, such as COVID-19 levels of infection in the neighborhood of the human wearing the smart mask or people contaminated with COVID-19 who have been within the proximity distance of the human wearing the smart mask

The microcontroller 210 may be connected to each of the plurality of sensors via a pin (1 TO 5) of the microcontroller 210, and the microcontroller 210 may be configured to power the electronic device via the corresponding pin. As such, an ON/OFF switch 252 is configured to connect the rechargeable battery 206 with the microcontroller 302 in the “ON mode” or disconnect the rechargeable battery 206 from the microcontroller 302 in an “OFF mode”. Additionally, the microprocessor may be configured to monitor the battery power level and send a message by Bluetooth communications to the smart phone 224 of the smart mask wearer that the battery must be recharged when the power level falls below a threshold value.

The communication device 210 includes, but is not limited to, the WiFi adaptor 213, which is based on the IEEE 802.11 standards, the Bluetooth adaptor 215, the GPS unit 228, and other devices capable of establishing wireless communication networks. With the aid of the communication device 220, the microcontroller 210 may be configured to receive data, analyze the data, and transmit the data to other electronic devices on the circuit board 250 or transmit the data WiFi access points or smartphone. Optionally, the microcontroller 210 may be configured to periodically generate a message including the location and time detected by the GPS unit 228 and transmit the message to the user's smartphone via the Bluetooth adaptor 215.

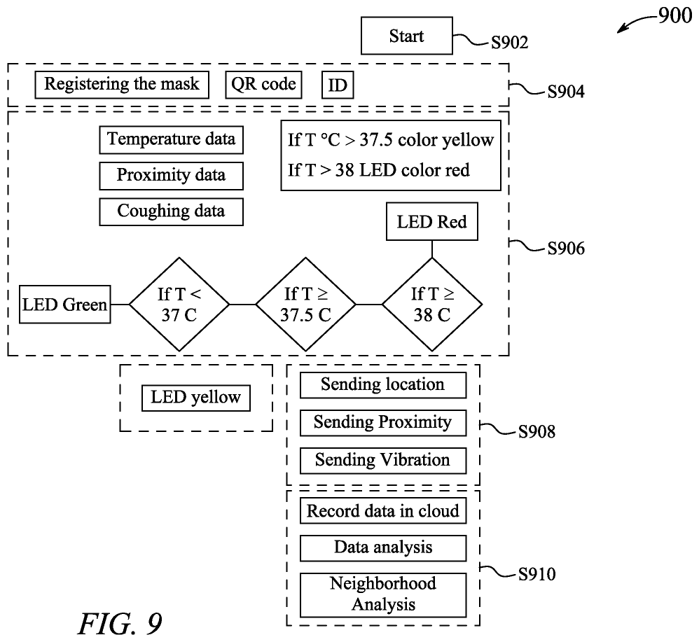

FIG. 9 illustrates the computing steps 900 of the cloud based smart mask monitoring application 216. At S902, monitoring starts. At S906, a message including sensor readings, the identification and the location of the smart mask 200 is received. At S904, the identification and is checked against an identification stored in a memory associated with the application. The smart mask is registered with the cloud based smart mask monitoring application 216 based upon reading QR code 230. At S906, the temperature, proximity and accelerometer readings are analyzed and a decision is made as to which LEDs should be lit. At S910 the readings are recorded in the memory, previous, historical readings are compared to the new readings of the smart mask, and a neighborhood analysis is performed to determine if other persons wearing smart masks are within the proximity of the human wearing the smart mask.

The above-described hardware description is a non-limiting example of corresponding structure for performing the functionality described herein.

Obviously, numerous modifications and variations of the present disclosure are possible in light of the above teachings. It is therefore to be understood that within the scope of the appended claims, the invention may be practiced otherwise than as specifically described herein.

1. A smart mask, comprising:
a transparent acrylic shield having a large opening configured to fit against a face and a small opening opposite the large opening;
an outer seal covering a circumference of the large opening;
a filter unit covering the small opening, the filter unit including a filter pocket;
a replaceable membrane located within the filter pocket, wherein the replaceable membrane is configured to filter airborne droplets;
an inner shield connected to the filter unit, wherein the inner shield is configured to surround a nose and a mouth of the human wearing the smart mask, and convey filtered air to the nose and mouth;
a plurality of LED lights located on an exterior of the transparent acrylic shield;
a plurality of sensors located on the transparent acrylic shield, each sensor configured to generate a sensor reading;
a communications unit configured for near field communications;
a global positioning system, GPS, unit configured to provide a geographic location of the smart mask;
a microcontroller located on the transparent acrylic shield, wherein:
the microcontroller is connected to the communications unit, the GPS unit and the plurality of sensors;
the microcontroller further includes an RFID tag configured to generate an identification signal;
the microcontroller is configured to generate a message including the sensor readings, the identification signal and the geographic location of the smart mask;
the microcontroller is configured to transmit the message to the communications unit; and
the communications unit is configured to transmit the message using near field communication.

2. The smart mask of claim 1, wherein the plurality of sensors comprise:
an accelerometer located on the cylindrical filter unit, wherein the accelerometer is configured to generate accelerometer readings;
a contactless infrared temperature sensor located on the transparent acrylic shield, wherein the contactless infrared temperature sensor is configured to generate forehead temperature sensor readings;
a tympanic ear canal temperature sensor located on the large opening of the transparent acrylic shield, wherein the ear canal temperature sensor is configured to generate ear canal sensor readings; and
a proximity sensor located on the large opening of the transparent acrylic shield, wherein the proximity sensor is configured to generate proximity sensor readings.

3. The smart mask of claim 2, wherein the accelerometer is configured to detect rapid movements of the smart mask.

4. The smart mask of claim 2, wherein the microcontroller is further configured to:
generate a first signal which activates a green LED when the ranges of the face temperature sensor readings and the ear canal temperature sensor readings are both below 37° C.;
generate a second signal which activates a yellow lighting indicator when the ranges of either of the face temperature sensor readings and the ear canal temperature sensor readings are greater than 37° C. and less than 37.5° C.; and
generate a third signal which activates a red lighting indicator when the ranges of the face temperature sensor readings and the ear canal temperature sensor readings are both greater than 37.5° C. and less than 38° C.

5. The smart mask of claim 2, wherein:
the smart mask is configured to be worn on a face of a first human; and
the proximity sensor is an ultrasound sensor which is configured to measure a distance from the first human to a second human within a distance in the range of zero meters to two meters.

6. The smart mask of claim 2, further comprising:
a WiFi adaptor located in the communications unit, the WiFi adaptor configured to adapt the near field communications to a WiFi protocol;
wherein the communications unit is further configured to:
locate a WiFi access point within range of the smart mask;
transmit the message to a WiFi access point using the WiFi adaptor;
instruct the WiFi access point to transmit the message to a cloud based smart mask monitoring application using an MQTT protocol;
receive an analysis of the sensor readings generated by the cloud based smart mask monitoring application, the analysis including whether the sensor readings indicate COVID-19 contamination of the smart mask, whether the proximity sensor readings indicate potentially contaminated second humans in a neighborhood of the first human, and whether the first human is contaminated by COVID-19 based on the face temperature sensor readings, the ear canal temperature sensor readings and accelerometer readings.

7. The smart mask of claim 6, wherein the analysis includes identifying when the rapid movements indicate one or more of sneezing and coughing of the first human.

8. The smart mask of claim 7, wherein the analysis includes a determination of a temperature range of the face temperature sensor readings and the ear canal temperature sensor readings, wherein a temperature below 37° C. indicates the first human is not contaminated with COVID-19, a temperature range of greater than 37° C. and less than 37.5° C. indicates the first human may be contaminated with COVID-19, and a temperature range of greater than 37.5° C. indicates the first human is contaminated with COVID-19.

9. The smart mask of claim 6, wherein the analysis includes a determination of a distance of at least one second human from the smart mask based on the proximity sensor readings;
wherein the microprocessor is further configured to:
generate a first signal which activates a green LED when second human is at a distance greater than 130 cm;
generate a second signal which activates a yellow LED when second human is at a distance greater than 100 cm and less than or equal to 130 cm; and
generate a third signal which activates a red lighting indicator when second human is at a distance less than or equal to 100 cm.

10. The smart mask of claim 2, further comprising:
a Bluetooth adaptor located within the communications unit, the Bluetooth adaptor configured to adapt the near field communications to a Bluetooth protocol;
wherein the communications unit is further configured to:
locate a smart phone belonging the human wearing the smart mask, wherein the smart phone equipped with a native smart mask monitoring application communicably connected to the cloud based smart mask monitoring application;
determine whether the smart phone is within ten meters of the communications unit;
transmit the message to the smart phone using the Bluetooth adaptor, wherein the native smart mask monitoring application is configured to perform an analysis of the sensor readings and the smart phone is configured to display the analysis; and
instruct the smart phone to transmit the message and the analysis to the cloud based smart mask monitoring application using an LTE protocol.

11. The smart mask of claim 10, further comprising:
a quick response, QR, code attached to the transparent acrylic shield, wherein the QR code is configured to identify the smart mask to the native smart mask monitoring application when the QR code is read by a QR code reader of the smart phone.

12. The smart mask of claim 1, wherein the identification signal is configured to identify the smart mask to the cloud based smart mask monitoring application hosted by a cloud server.

13. The smart mask of claim 1, further comprising:
wherein the inner shield is a silicone material; and
the seal is a silicone seal configured to seal the large opening to an outer perimeter of a human face.

14. A method for protecting humans from COVID-19, comprising:
covering a face of a first human with a smart mask, wherein the smart mask is configured to filter air entering a mouth and nose of the face;
sending, by a proximity sensor, a proximity sensor signal to a microcontroller located on the smart mask when a proximity sensor detects an object within one meter of the first human;
measuring, with a tympanic ear canal thermometer, an ear canal temperature of the first human, generating an ear canal temperature signal and transmitting the ear canal temperature signal to the microcontroller;
detecting, with an accelerometer, rapid movements of the smart mask and sending an accelerometer signal to the microcontroller;
detecting a face temperature by a contactless infrared temperature sensor located on the smart mask, and sending a face temperature signal to the microcontroller;
generating, by a global positioning system, GPS, unit operatively connected to the microcontroller, a geographic location of the smart mask;
generating, by an RFID tag operatively connected to the microcontroller, an identification signal; and
transmitting, by a communications unit operatively connected to the microcontroller, a message including the proximity sensor signal, the ear canal temperature signal, the accelerometer signal, the face temperature signal, the geographic location and the identification signal by a near field communication protocol to a WiFi access point; and
transmitting, by the WiFi access point, the message to a cloud server hosting a cloud based smart mask monitoring application using an MQTT protocol.

15. The method of claim 14, further comprising:
averaging, by the cloud based smart mask monitoring application, the face temperature sensor signals and the ear canal temperature sensor signals;
comparing, by the cloud based smart mask monitoring application, the average temperature to a set of threshold temperatures;
generating, by the cloud based smart mask monitoring application, instructions for lighting a set of LEDs on the smart mask;
transmitting, by the cloud based smart mask monitoring application, the instructions to the WiFi access point using the MQTT protocol;
transmitting, by the WiFi access point, the instructions to the communications device using the near field communication protocol;
generating, by the microcontroller based on the instructions, a first signal which activates a green lighting indicator when the average temperature is below 37° C.;
generating, by the microcontroller based on the instructions, a second signal which activates a yellow lighting indicator when the average temperature is greater than 37° C. and less than 37.5° C.; and
generating, by the microcontroller based on the instructions, a third signal which activates a red lighting indicator when the average temperature is greater than 37.5° C. and less than 38° C.

16. The method of claim 14, further comprising:
receiving, by the microprocessor, an analysis of the sensor signals from the cloud based smart mask monitoring application, the analysis including whether the sensor signals indicate COVID-19 contamination of the smart mask, whether the proximity sensor signals indicate potentially contaminated second humans in a neighborhood of the first human, and whether the first human is contaminated by COVID-19 based on the face temperature sensor signals, the ear canal temperature sensor signals and accelerometer signals.

17. The method of claim 16, further comprising:
determining, by the face monitoring application, whether the rapid movements indicate sneezing or coughing of the first human;
determining, by the face monitoring application, a temperature range of the face temperature sensor signals and the ear canal temperature sensor signals;
when the first human is not sneezing or coughing and the temperature range is below 37° C., reporting in the analysis that the first human is not contaminated with COVID-19;
when the first human is sneezing or coughing and the temperature range is greater than 37° C. and less than 37.5° C., reporting in the analysis that the first human may be contaminated with COVID-19; and
when the first human is sneezing or coughing and the temperature range is greater than 37.5° C., reporting in the analysis that the first human is contaminated with COVID-19.

18. The method of claim 17, further comprising:
transmitting the analysis, by the communications unit by using a Bluetooth communications protocol, to a smart phone equipped with a native smart mask monitoring application, wherein the smart phone is configured to scan, using a scanner, a QR code attached to the smart mask, identify the smart mask by the QR code, and display the analysis.

19. A cloud based smart mask monitoring application configured to detect viral exposure of a human wearing a smart mask, comprising:
receiving, by the cloud based smart mask monitoring application, a communications packet including proximity sensor signals, ear canal temperature signals, accelerometer signals, face temperature signals, a geographic location signal and a smart mask identification signal;
analyzing the communications packet to detect viral exposure of a first human wearing the smart mask;
when detecting viral exposure of the first human:
identifying, from the proximity sensor signals, whether objects within a proximity of the protective face are one or more second humans;
generating an analysis report of the viral exposure of the first human;
transmitting the report to a native face mask application stored on a smart phone of the first human; and
alerting the one or more second humans of the danger of viral contamination by one or more of calling the one or more second humans, texting the one or more second humans, emailing the one or more second humans, and activating a lighting indicator on the smart mask worn by the first human.

20. The method of claim 19, further comprising:
determining, by the face monitoring application, whether the rapid movements indicate sneezing or coughing of the first human;
determining, by the face monitoring application, an average temperature of the face temperature sensor signals and the ear canal temperature sensor signals;
when the first human is not sneezing or coughing and the average temperature is below 37° C., reporting in the analysis that the first human is not contaminated with COVID-19;
when the first human is at least one of sneezing and coughing and the average temperature is greater than 37° C. and less than 37.5° C., reporting in the analysis that the first human may be contaminated with COVID-19; and
when the first human is at least one of sneezing and coughing and the average temperature is greater than 37.5° C., reporting in the analysis that the first human is contaminated with COVID-19.
